(19) United States
(12) Patent Application Publication
(10) Pub. No.: US 2005/0261239 A1
(43) Pub. Date: Nov. 24, 2005

(54) Use of ATP for the manufacture of a medicament for treating certain inflammatory conditions, oxidative stress and fatigue

(75) Inventors: Pieter Cornelis Dagnelie, Banholt (NL); Els Louise Rita Swennen, Tongeren (BE)

Correspondence Address: KALOW & SPRINGUT LLP, 488 MADISON AVENUE, 19TH FLOOR, NEW YORK, NY, 10022, US

(73) Assignee: Universiteit van Maastricht, Maastricht (NL)

(21) Appl. No.: 10/903,626

(22) Filed: Jul. 30, 2004

(30) Foreign Application Priority Data
May 23, 2004 (EP) 04076502.6

(51) Int. Cl. A61K 31/7076

(52) U.S. Cl. 514/48

(57) ABSTRACT

The present invention provides the use of ATP for the manufacture of a medicine for exerting a pharmacological effect when administered to a mammal, preferably a human, selected from the group consisting of: 1°. modulating inflammation by inhibiting the inflammatory response to a strong external insult such as endotoxin (LPS) and/or phytohaemagglutinin; 2°. exerting said inhibitory effect on inflammatory response to an external stimulus even under conditions of oxidative stress, 3°. exerting a local immuno-modulating and anti-inflammatory effect in the intestine, thus preventing intestinal damage induced by a non-steroid anti-inflammatory drug (NSAIDs), 4°. exerting an immuno-modulating and anti-inflammatory effect in human intestinal cells in vitro, 5°. alleviating pulmonary symptoms, such as shortness of breath and dyspnoea, in patients suffering from an obstructive pulmonary disease, and 6°. exerting favorable clinical effects with respect to certain mental and neurological disorders and aberrant conditions. The medicine is preferably manufactured in lyophilized form.

### FIELD OF THE INVENTION

The present invention relates to the use of adenosine 5′-triphosphate in the prevention and treatment of certain inflammatory disorders including conditions of aberrant, excessive, depressed, or insufficient immune response, oxidative stress and fatigue in mammals, in particular humans. Furthermore, the invention relates to a novel method of manufacturing a formulation comprising ATP which greatly facilitates ATP administration in a non-medical setting, such as in private homes, nursing homes etc.

### BACKGROUND OF THE INVENTION

a. Related Art

Adenosine 5′-triphosphate (ATP) is a naturally occurring nucleotide which is present in every cell. Nucleotides were first recognised as important substrate molecules in metabolic interconversions, and later as the building blocks of DNA and RNA. More recently, it was found that nucleotides are also present in the extracellular fluid under physiologic circumstances. The prior art concerning the physiology and established and potential clinical applications of ATP, as well as its pharmacokinetic properties, physiological effects and mechanisms of action has been reviewed (1).

ATP has recently aroused interest because of its properties as a signaling substance outside the cell (extracellular ATP). Extracellular ATP is known to be involved in the regulation of a variety of biological processes including neurotransmission, muscle contraction, cardiac function, platelet function, and vasodilatation.

ATP can be released from the cytoplasm of several cell types and interacts with specific purinergic (receptors which are present on the surface of many cells and play a fundamental role in cell physiology. Intravenous administration of ATP induces a rapid rise in ATP levels uptake by erythrocytes (2) and liver (3) followed by slow release into the plasma compartment.

In the past years, possible pharmacological uses of ATP have received attention, following reports of its potential benefit in pain, vascular diseases and cancer. ATP has cytostatic and cytotoxic effects in many types of transformed and tumour cells (for review, see (1)). Several mechanisms have been proposed, including: 1. intracellular accumulation of ATP and arrest of tumour cells in the S-phase of cell replication, followed by cell death (4, 5); 2. intracellular adenosine leading to elevation of ATP and ADP levels and reduction of uridine 5′-triphosphate (UTP) concentrations, inducing inhibition of pyrimidine nucleotide biosynthesis (6, 7); and 3. Reduction in glutathione content of the tumour (7, 8).

In vivo daily intraperitoneal injections of 25 mmol/L ATP, AMP or adenosine for 10 consecutive days into mice bearing colon tumour induced a significant inhibition of host weight loss in this experimental cancer model (9). This inhibition was associated with expansion of erythrocyte ATP pools (10).

In the USA, a phase I/II trial was carried out in 8 stage IIIB/IV patients with non-small cell lung cancer. After treatment with 2 to 3 intravenous ATP courses of 96 hours at 4-week intervals, stabilisation of body weight was observed (11). In a subsequent open-ended phase II trial in 15 newly diagnosed patients with non-small cell lung cancer, an average weight gain of 1.3 kg was demonstrated after 4 ATP courses (12).

In a randomized clinical trial in advanced non-small-cell lung cancer patients (13), it was shown that regular infusions of adenosine 5′-triphosphate (ATP) inhibited loss of weight and muscle mass compared to a control group of non-small-cell lung cancer patients (stage IIIB or IV) receiving usual non-small cell lung cancer supportive care only. Moreover, physical and functional quality of life, appetite, and muscle strength remained stable in the ATP group, but progressively deteriorated in the control group. Although preliminary data from a small subset of cancer patients suggested potential inhibition of C-reactive protein by ATP, further analyses showed no effect of ATP on plasma levels of pro- or anti-inflammatory cytokines in this patient population (Dagnelie et al. 2003, unpublished data).

Insight into the role of ATP in immunity and inflammation derives mainly from in vitro studies, and a relatively small number of animal studies in vivo. By stimulating purinergic receptors, ATP (and adenosine) exert various effects on different cell types involved in the immune response such as neutrophils, monocytes/macrophages, lymphocytes, dendritic cells, microglial cells, mast cells and endothelial cells. These studies have in general been performed using cultured cells or cell lines derived from humans or animals, and are directed towards unravelling biochemical mechanisms at molecular receptor and post-receptor levels, rather than providing a realistic picture of normal physiological or pathological situations in human subjects in vivo. Generally speaking, such studies in cultured cell lines and isolated cells are far away from the in vivo situation for a number of reasons. One such reason is that, due to repeated cell divisions, cell lines develop features which are distinct from in vivo human cells, for instance with respect to receptor expression and activity, intracellular cascades, transcription factors, etc. Furthermore, cell-to-cell interactions between different cell types, which play an essential role in determining physiological effects in the in vivo situation, are absent.

Based on these studies, it is generally thought that ATP has a pro-inflammatory role in the immune system, whereas adenosine has a more anti-inflammatory role. For instance, ATP stimulates chemotaxis of macrophages and dendritic cells (14), thereby contributing to migration of leukocytes to sites of inflammation. At these inflammatory sites ATP stimulates adhesion of neutrophils, monocytes and macrophages to the vascular endothelium by up-regulation of adhesion molecules (15, 16). ATP also promotes leukocyte phagocytosis by enhancing degranulation and the release of reactive oxygen and nitrogen species by neutrophils and macrophages (14-16). Neutrophils stimulated by ATP also release arachidonic acid, eventually leading to the formation of leukotriene B4 which attracts even more neutrophils to sites of inflammation. Cytokines such as TNF-α, interleukin (IL)-1β, IL-6, IL-8 and IL-18 promote inflammatory processes by increasing the production of other cytokines, attracting more leukocytes to sites of inflammation and activating the leukocytes that are already present. The increased production of inflammatory mediators partially results from activation of certain transcription factors such as NFκB or AP-1 by ATP involved in the production of these inflammatory mediators (14). ATP can also form large pores in the cell membranes of various immune cell types, which may contribute to cell-to-cell communication. The direct effects of ATP on the immune system according to the state of the art can be summarized as pro-inflammatory, and are thought to be aimed at activating the immune system at the initial stage of inflammation.

The patent literature also reveals a variety of new applications and further developments relating to adenosine triphosphate (ATP) and other adenosine derivatives including adenosine.

For example, EP 0 352 477 of Rapaport discloses the use of AMP, ADP and ATP in the treatment of cancer-related cachexia.

U.S. Pat. No. 4,880,918 and U.S. Pat. No. 5,049,372 to Rapaport disclose anticancer activities (i.e. inhibition of the growth of tumor cells) in a host by increasing blood and plasma ATP levels.

U.S. Pat. No. 5,227,371 to Rapaport discloses the administration of AMP, ATP or their degradation products adenosine and inorganic phosphate to a host, achieving the beneficial increases in ATP levels in liver, total blood and blood plasma.

U.S. Pat. No. 5,547,942 to Rapaport discloses the administration of ATP or other adenine nucleotides and inorganic phosphates to human patients in treating non-insulin-dependent diabetes mellitus following the interactions of extracellular ATP pools with pancreatic beta cell purine receptors.

U.S. Pat. No. 6,159,942 to St. Cyr et al. discloses the oral administration of precursors of ATP, in particular pentose sugars such as D-ribose, to increase intracellular ATP concentration as dietary supplements or for treatment of reduced energy availability resulting from strenuous physical activity, illness or trauma.

U.S. 2003/0109486 of Rapaport discloses methods for the utilization of ATP in the treatment of acute lung injury (ALI) and acute respiratory distress syndrome (ARDS). The administration of ATP to patients at intensive care units who suffer from these specific conditions is said to result in several therapeutic activities which by acting in consort provide the methods for treatment of ALI and ARDS. The following therapeutic activities are claimed to occur in these life-threatening conditions: 1. the utility of ATP as a preferential pulmonary vasodilator, 2. the utility of the catabolic product of ATP, adenosine, as an anti-inflammatory agent, 3. the anti-thrombotic, pro-fibrinolytic activities of ATP and adenosine, and 4. the utility of ATP in improving organ and muscle function in advance disease patients.

WO 01/028528 of Rapaport discloses methods for preventing/reducing weight gain by administering ATP in coated form for the chronic administration of adenosine, aiming at desentisizing A1 adenosine receptors towards the action of adenosine and thereby increasing intracellular levels of cyclic AMP, thereby resulting in stimulation of lipolysis.

U.S. 2003/0069203 of Lee et al. discloses a composition for oral administration used for improving muscle torque and reducing muscle fatigue comprising an effective amount of ATP in an enteric coating that protects ATP from degradation by gastric juices, to enhance absorption into the blood stream and provide additional therapeutic benefit.

b. Prior Knowledge Regarding Intravenous ATP Administration in Humans

In all reports published to date, intravenous ATP administration was performed under strict medical supervision, either at a medical ward or in a day care center of the hospital, because of the adherent risk of potential side effects of ATP. However, there are several major limitations to the application of ATP administered in such a hospital setting:
The regular stays at the hospital ward or day care center for ATP infusions (e.g. once per 1-4 weeks) comprise a considerable burden to patients,
These ATP infusions put a high demand on scarce resources of hospital beds and specialized medical care,
And cause high costs for the health care system.

For reasons of patients' safety, there has been no attempt to administer ATP outside a strict medical setting to date. In particular, WO 03/061568 (Rapaport) discloses the administration of ATP over a period of typically 8-10 hours in an outpatient setting within the hospital. The patent specification is allegedly based on the observation that short, weekly, continuous infusions of ATP, “at infusion rates even somewhat higher than what has been previously reported”, resulted in similar clinical efficacies with significantly reduced profiles of adverse effects compared to longer (30-96 hrs) infusions. However, our experience with over 200 ATP infusions varying in dose (25-75 μg/kg.min) and duration (8-30 hrs) demonstrates that side effects induced by intravenous ATP infusion only depend on the infusion rate, and not on the duration of the ATP infusions. Moreover, in contrast with quotations of our work in the aforementioned patent application, we did not find any life-threatening side effects in our previous study with ATP infusion during 30 hrs (13), as is correctly quoted in another patent application by Rapaport (U.S. 2003/0109486 on utilization of ATP in treatment of ALI/ARDS). Thus, our data and U.S. 2003/0109486 contradict the disclosure of WO 03/061568.

There is a continuous interest in exploring possible further pharmacological uses of ATP and ways of administering ATP because of its favorable properties hitherto known. The present invention provides some new uses of this substance with promising results, as well as novel ways and methods for facilitating the administration of ATP without direct medical supervision, e.g. at private homes, nursing homes, etc.

### SUMMARY OF THE INVENTION

It has now been surprisingly found, after extensive research and testing, that ATP 1°. modulates inflammation by inhibiting the inflammatory response to a strong external insult such as endotoxin (LPS) and/or phytohaemagglutinin; 2°. exerts this inhibitory effect on inflammatory response to an external stimulus even under conditions of oxidative stress, 3°. exerts a local immuno-modulating and anti-inflammatory effect in the intestine, thus preventing intestinal damage induced by non-steroid anti-inflammatory drugs (NSAIDs), 4°. exerts immuno-modulating and anti-inflammatory effects in human intestinal cells in vitro, 5°. alleviates pulmonary symptoms such as shortness of breath and dyspnoea in patients suffering from obstructive pulmonary diseases, and 6°. exerts favorable clinical effects with respect to certain mental and neurological disorders and aberrant conditions.

Therefore, in a first aspect the present invention provides the use of ATP for the manufacture of a medicine for exerting a pharmacological effect when administered to a mammal, preferably a human, selected from the group consisting of:
1°. modulating inflammation by inhibiting the inflammatory response to a strong external insult such as endotoxin (LPS) and/or phytohaemagglutinin;
2°. exerting said inhibitory effect on inflammatory response to an external stimulus even under conditions of oxidative stress,
3°. exerting a local immuno-modulating and anti-inflammatory effect in the intestine, thus preventing intestinal damage induced by a non-steroid anti-inflammatory drug (NSAIDs),
4°. exerting an immuno-modulating and anti-inflammatory effect in human intestinal cells in vitro,
5°. alleviating pulmonary symptoms, such as shortness of breath and dyspnoea, in patients suffering from an obstructive pulmonary disease, and
6°. exerting favorable clinical effects with respect to certain mental and neurological disorders and aberrant conditions.

In a further aspect of the present invention, the use of ATP is provided for the manufacture of a medicine comprising ATP as an active ingredient having an preventive or curative activity when administered to a mammal, preferably a human, selected from the group consisting of:
1°. tissue-protecting activity which attenuates excessive inflammation under varying conditions of oxidative stress and inflammation;
2°. immune-stimulating activity under varying conditions related to immune-incompetence and immuno-suppression;
3°. immuno-modulating activity normalizing the Th1/Th2 balance in aberrant conditions of aberrant Th2-skewed immune response, such as atopic diseases and asthma, as well as in conditions of aberrant Th1-skewed response, such as auto-immune disorders;
4°. modulating and normalizing aberrant mental and neurological states and diseases.

In still a further aspect of the present invention the use of ATP is provided for the manufacture of a medicine wherein the medicine is for preventing or treating at least one of intestinal inflammatory condition, intestinal damage, and inflammatory bowel disease.

In yet another aspect of the present invention the use of ATP is provided for the manufacture of a medicine wherein the medicine is for preventing or treating rheumatoid arthritis.

In a further aspect of the present invention the use of ATP is provided for the manufacture of a medicine wherein the medicine is for preventing or treating atopic disease, including asthma.

In another aspect of the present invention the use of ATP is provided for the manufacture of a medicine wherein the medicine is for preventing or treating a condition selected from the group consisting of fatigue, fibromyalgia, burn-out and depression.

In still a further aspect of the present invention the use of ATP is provided wherein the medicine is for preventing or treating an individual for a disease or disorder or condition selected from the group consisting of intestinal inflammation, intestinal damage, rheumatoid arthritis, COPD, cancer during or after treatment by at least one of surgery, radiotherapy, and chemotherapy, a neurological or mental disorder, an atopic disease including asthma, and another condition of elevated or aberrant inflammatory response, for example an auto-immune disorder, disease and condition of immunosuppression and immuno-incompetence, or limited resistance towards infections.

In yet another aspect of the invention a method is provided of preventing or treating an individual for a disease or disorder or condition selected from the group consisting of intestinal inflammation, intestinal damage, rheumatoid arthritis, COPD, cancer during or after treatment by at least one of surgery, radiotherapy, and chemotherapy, a neurological or mental disorder, an atopic disease including asthma, and another condition of elevated or aberrant inflammatory response, which comprises administering to said individual in need thereof a medicine comprising an effective amount of ATP.

In a preferred embodiment of the invention the medicine is in the form of a pharmaceutical composition or a nutritional composition, and is most preferably in a lyophilized form.

These and other aspects of the invention will be discussed in more detail below.

### BRIEF DESCRIPTION OF THE DRAWINGS

FIG. 1 Effect of ATP on LPS+PHA-induced TNF-α secretion in whole blood from healthy subjects. The whole blood was exposed to 10 μg/ml LPS and 1 μg/ml PHA with indicated concentrations of ATP for 24 h. The TNF-α released into the supernatants was analyzed using the ELISA method. Results are expressed in percentage, 100% being the TNF-α release under stimulation by LPS+PHA without ATP. The TNF-α release induced by LPS+PHA from whole blood was significantly inhibited by the addition of ATP. Data are expressed as the mean values; error bars represent SEM. *, different from control (stimulation by LPS+PHA without ATP) (P<0.05).

FIG. 2 Effect of ATP on LPS+PHA-induced IL-10 secretion in whole blood from healthy subjects. The whole blood was exposed to 10 μg/ml LPS and 1 μg/ml PHA with indicated concentrations of ATP for 24 h. The IL-10 released into the supernatants was analyzed using the ELISA method. Results are expressed in percentage, 100% being the IL-10 release under stimulation by LPS+PHA without ATP. The IL-10 release induced by LPS+PHA from whole blood was significantly increased by the addition of ATP. Data are expressed as the mean values; error bars represent SEM. *, different from control (stimulation by LPS+PHA without ATP) (P<0.05).

FIG. 3 Effect of ATP on LPS+PHA-induced IL-6 secretion in whole blood from healthy subjects. The whole blood was exposed to 10 μg/ml LPS and 1 μg/ml PHA with indicated concentrations of ATP for 24 h. The IL-6 released into the supernatants was analyzed using the ELISA method. Results are expressed in percentage, 100% being the IL-6 release under stimulation by LPS+PHA without ATP. The IL-6 release induced by LPS+PHA from whole blood was not influenced by the addition of ATP. Data are expressed at the mean values; error bars represent SEM.

FIG. 4 Relationship between TNF-α and IL-10 secretion in LPS+PHA stimulated whole blood in healthy subjects. The inhibition of the TNF-α release by ATP is related to the stimulation of the IL-10 release by ATP. The figure shows the cytokine release at 300 μM ATP, expressed as percentage of control (stimulation by LPS+PHA without ATP).

FIG. 5 Effect of ATP on LPS+PHA-induced TNF-α secretion in whole blood from healthy subjects under conditions of oxidative stress. Two concentrations of H₂O₂ (1 and 10 mM) were added to whole blood, together with the indicated concentrations of ATP. Then, blood was exposed to 10 μg/ml LPS and 1 μg/ml PHA, and incubated for 24 h. The TNF-α released into the supernatants was analyzed using the ELISA method. The TNF-α release induced by LPS+PHA from whole blood was significantly inhibited by the addition of ATP. Data are expressed as the mean values; error bars represent SEM.

FIG. 6 Effect of ATP on LPS+PHA-induced IL-10 secretion in whole blood from healthy subjects under conditions of oxidative stress. Two concentrations of H₂O₂ (1 and 10 mM) were added to whole blood, together with the indicated concentrations of ATP. Then, blood was exposed to 10 μg/ml LPS and 1 μg/ml PHA, and incubated for 24 h. The IL-10 released into the supernatants was analyzed using the ELISA method. The IL-10 release induced by LPS+PHA from whole blood was significantly increased by the addition of ATP. Data are expressed as the mean values; error bars represent SEM.

FIG. 7 Effect of ATP on LPS+PHA-induced IL-6 secretion in whole blood from healthy subjects under conditions of oxidative stress. Two concentrations of H₂O₂ (1 and 10 mM) were added to whole blood, together with the indicated concentrations of ATP. Then, blood was exposed to 10 μg/ml LPS and 1 μg/ml PHA, and incubated for 24 h. The IL-6 released into the supernatants was analyzed using the ELISA method. The IL-6 release induced by LPS+PHA from whole blood was not influenced by the addition of ATP. Data are expressed at the mean values; error bars represent SEM.

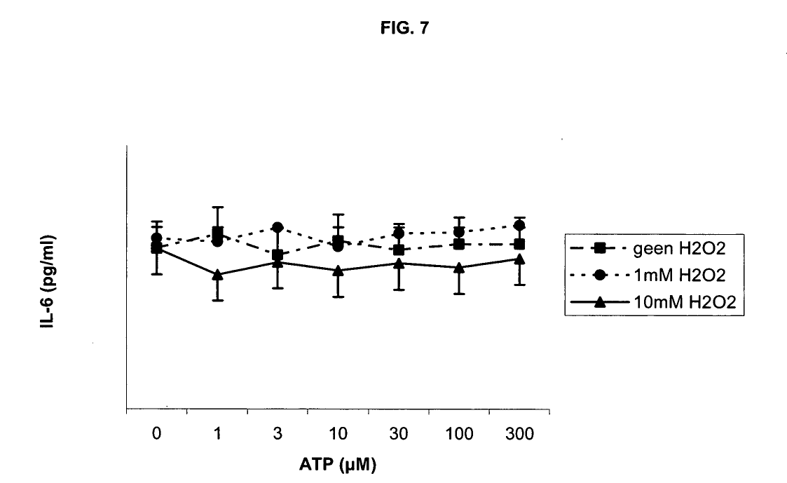

FIG. 8 Effect of different purinergic compounds on LPS+PHA-induced TNF-α secretion in whole blood. The whole blood is exposed to 10 μg/ml LPS and 1 μg/ml PHA with the indicated purinergic compounds at the concentration of 300 μM for 24 h. The TNF-α released into the supernatants is analyzed using the ELISA method. Results are expressed in percentage, 100% being the TNF-α release under stimulation by LPS+PHA without addition of a purinergic compound. The TNF-α release induced by LPS+PHA from whole blood is inhibited by different compounds in the following order: adenosine (least inhibition) <AMP<ADP<ATP (greatest inhibition). The TNF-α release induced by LPS+PHA from whole blood is not inhibited by either UTP, UDP or CTP. Data are expressed as the mean values; error bars represent SEM.

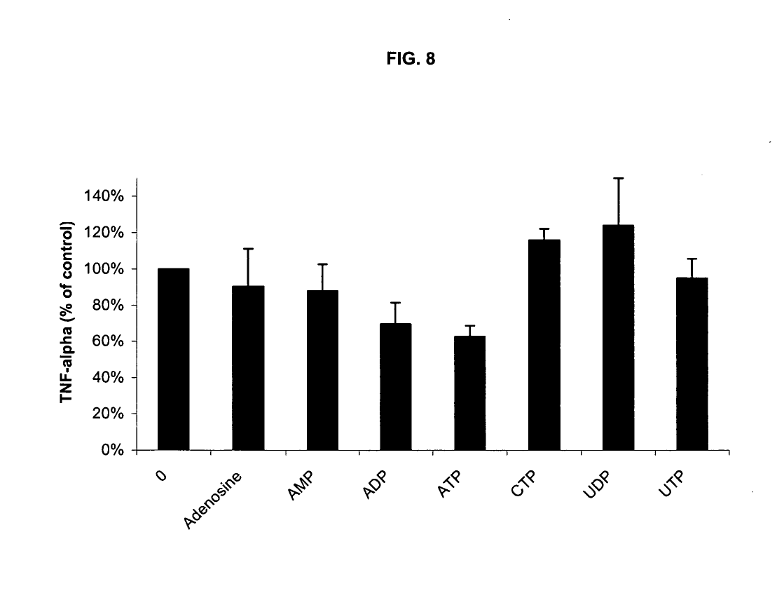

### DETAILED DESCRIPTION OF THE INVENTION

As used herein, the term “ATP” is meant to include also related compounds or substances that are functionally equivalent with ATP, i.e. with a substantially similar profile of effect in inflammatory processes as herein described, as well as pharmacologically acceptable salts thereof, or chelates thereof, or metal cation complexes thereof, or liposomes thereof, or incorporated in particles, e.g. for specific purposes such as drug targeting, in magnetic particles, incorporated in polymers such as DNA or RNA, etc. Examples of such related compounds or substances include analogues, derivatives and metabolites of ATP (including natural and synthetic compounds) that are functionally equivalent, for example purine and pyrimidine nucleotides such as UTP, GTP, CTP. Also included is a functionally equivalent combination of adenosine, AMP, and ADP, respectively, with phosphate, preferably inorganic phosphate. For particulars of such a combination, reference is made to refs. 9 and 10 the contents of which are herewith incorporated by reference.

The present invention is predominantly based on the empirical observation that ATP down-regulates the expression of pro-inflammatory cytokines such as TNF-alpha and up-regulates the expression of anti-inflammatory cytokines such as IL-10 in different circumstances, and that this effect is pertained even under conditions of oxidative stress.

As indicated above, we have now found that ATP inhibits excessive inflammation by inhibiting the inflammatory response to an external insult such as endotoxin (LPS) or phytohaemagglutinin (PHA). Results from experiments in a model study show for the first time that ATP in whole blood inhibits the inflammatory response to a strong inflammatory insult such as LPS and PHA, thus modulating the cytokine production in whole blood.

In addition, we found that ATP inhibits excessive inflammation by inhibiting the inflammatory response to an external insult, such as LPS and PHA, even under circumstances of oxidative stress. The results from our experiments which will be detailed hereinafter show for the first time that ATP inhibits the inflammatory response to a strong inflammatory insult such as LPS and PHA in the presence of oxidative stress, by modulating the cytokine production in whole blood.

It was also found that the anti-inflammatory effects of ATP are stronger than those of adenosine, AMP and ADP in the same model. It was further found in the same model as used above that the effects of ATP can be at least partly mimicked by incubating whole blood ex vivo with P2 receptor agonists, such as 2-MeS-ATP, instead of ATP.

Furthermore, we found that ATP reduces the intestinal permeability induced by NSAIDs in the small intestine of human subjects, as assessed by the lactulose/rhamnose (L/R) intestinal permeability test. The effect of ATP is believed to be stronger than that of adenosine.

Furthermore, it was found that ATP exerts certain new surprising favourable clinical effects in patients with advanced cancer, which are related to mental state, mood and neurological functioning, such as dry/sore mouth, worrying, dizziness, decreased sexual interest, tension, and sleeping difficulties.

Thus, in preventing and treating certain clinical conditions and diseases, ATP can inter alia be used in the framework of the present invention in conditions associated with inflammation in any part of the body, such as intestinal inflammation, inflammatory bowel disease, rheumatoid arthritis, etc., as well as in conditions of immunosuppression as caused by diseases such as acquired immunity deficiency syndrome (AIDS) or by immunosuppressive medication, in conditions of aberrant Th1- or Th2-skewed immune response, and in conditions of aberrant mental and neurological states and diseases.

Before our findings and their practical use will be further detailed, a brief survey is given of the various disorders in which in accordance with the present invention ATP shows, or, alternatively, is expected to possess some beneficial effects.

Inflammatory Diseases/Conditions, and Oxidative Stress

It is now generally accepted that many chronic diseases and conditions in mammals and humans are characterized by an increased or aberrant inflammatory response and elevated oxidative stress caused by, amongst others, reactive oxygen species. Well-known inflammatory diseases and conditions include, amongst many others, inflammatory bowel disease (IBD), rheumatoid arthritis (RA), and chronic obstructive pulmonary disease (COPD). However, over the last decades, it has been increasingly recognized that an elevated inflammatory response also plays an essential role in many other diseases and conditions, including, for example, the peri-operative inflammatory response, trauma, the systemic inflammatory distress syndrome (SIRS), acute and chronic cardiovascular diseases, atherosclerosis, heart failure, ischaemia-reperfusion, diabetes, syndrome X, obesity, wasting conditions such as cachexia and sarcopenia with loss of lean body mass, muscle mass, muscle strength and/or fat mass, osteoporosis, fibromyalgia, infectious diseases, and inflammatory pain syndromes. Aberrant inflammatory responses also play a role subsequent to treatment with drugs. For example, in the intestine, local inflammation is a frequent side effect of oral non-steroid anti-inflammatory drugs (NSAIDs). Furthermore, atopic diseases including allergic rhinitis, atopic dermatitis, vernal conjunctivitis and asthma are attributed to an aberrant and elevated inflammatory response; and finally, several conditions with a behavioral component, such as sickness behavior, fatigue and some eating disorders such as anorexia, are now considered to be associated with an elevated inflammatory response. A full discussion of these conditions is far beyond the scope of the current patent application; however, some of these conditions will be briefly discussed below to illustrate the human and societal impact of inflammatory disorders.

Inflammatory bowel diseases (IBD, e.g. ulcerative colitis and Crohn's disease) are characterized by chronic intestinal inflammation (17), with typically episodic relapses between longer spontaneous or treatment-induced remissions. Patients with active disease may present with diarrhea, abdominal pain, weight loss, anorexia and fatigue. The incidence of IBD in northern Europe is approximately 200 per million per year. The pathogenesis of IBD appears to involve the interaction between environmental factors and genetic susceptibility, which leads to immune-driven inflammation in the gut mucosa. Thus, Crohn's disease is associated with a Th1-type immune response with excessive production of TNF-α. The excessive production of pro-inflammatory mediators and reactive oxygen species, which cause oxidative stress (18), perpetuate the inflammatory reaction and may result in tissue damage and increased intestinal permeability.

Rheumatoid arthritis (RA) is a progressive disease of unknown etiology which involves features of both acute and chronic inflammatory processes in multiple joints. Inflammatory cells such as neutrophils, macrophages and lymphocytes invade the synovium of these joints and produce a variety of inflammatory mediators contributing to the progression of inflammatory processes. Recent findings indicate that increased oxidative stress contributes to the etiology of RA (19). Ongoing inflammation of multiple joints eventually causes bone destruction and joint deformities. RA patients also frequently suffer from fatigue as well as from loss of weight, appetite and general performance. RA constitutes a major public health problem, which affects about 1 percent of the general population worldwide and, due to its chronic and invalidating character, causes a tremendous burden on health care budgets.

In current medical practice, a number of problems face patients with diseases such as IBD and RA:
In many patients, the efficacy of current anti-inflammatory drugs (including novel drugs such as blockers of tumor necrosis factor alpha (TNF-α) only lasts for a limited period; some patients do not respond to such drugs at all.
Also, many patients develop side effects of medication which either compromises their quality of life, or necessitates them to stop using these drugs.
Novel medications such as TNF blockers are extremely costly which limits their long-turn use for reasons of health care costs.

These problems indicate a need for complementary treatment modalities which are effective, cheap and without side effects.

Non-steroidal anti-inflammatory drugs (NSAIDs), e.g. indomethacin, naproxen, ibuprofen, are among the most prescribed anti-inflammatory and analgesic drugs worldwide. However, paradoxically, the use of NSAIDs is associated with an elevated risk of mucosal damage and local inflammation in the gastrointestinal tract which can eventually result in pathological conditions such as perforations, ulcers or strictures (20). Specific detrimental effects of NSAIDs in the small intestine occur in approximately 70% of the patients who chronically take NSAIDs; on discontinuation of NSAIDs, such effects may persist for up to 16 months. Disruption of the intestinal barrier function is thought to contribute to the pathogenesis of several intestinal and systemic diseases, including coeliac disease and inflammatory bowel disease.

Asthma and chronic obstructive pulmonary disease (COPD) are widespread respiratory problems worldwide. For instance, in the United States only, the prevalence of COPD, which encompasses chronic obstructive bronchitis and emphysema, has been estimated as high as 30-35 million cases, and it is the fourth most common cause of death. Characteristic to both asthma and COPD is difficulty in breathing, mucus hypersecretion in the lungs, and cough. Part of the condition is an abnormal inflammatory response in the lung, which suggests the need for effective anti-inflammatory treatment in COPD. Over the past 30 years, despite the steady rise in occurrence of COPD, few new significant therapeutic modalities for the treatment of these disorders have been introduced in the clinical setting (21).

Cellular production of adenosine, the breakdown product of ATP, is greatly enhanced under conditions of local hypoxia as may occur in inflammatory conditions such as asthma and COPD. In patients with asthma and COPD, but not in healthy volunteers, Inhaled adenosine induces dose-related bronchoconstriction. Adenosine may also be involved in exercise-induced bronchoconstriction in asthmatic patients. Based on these notions, it was suggested that pharmaceutical agents that can block these actions of ATP could constitute a new therapeutic modality in the management of asthma and COPD (21, 22).

There has been a significant increase in the prevalence of allergic diseases over the past decades. Currently, it is estimated that more than 130 million humans worldwide suffer from asthma, and the numbers are increasing. Nevertheless, there is a considerably lower prevalence of allergic diseases in developing countries, as well in rural relative to urban areas. Among environmental factors, childhood infections show a consistent negative association with atopy and allergic diseases. Atopy, characterized by raised immunoglobulin (IgE) levels, underlies allergic diseases such as asthma, allergic rhinitis (hay fever), and atopic dermatitis (eczema). The initial sensitization to environmental allergens occurs typically in childhood. It is currently thought that a skewed inflammatory response from Th1 toward Th2 lymphocytes is a fundamental underlying mechanism of atopy and allergic disease. Recent evidence suggests that another newly discovered class of T-lymphocytes, the regulatory T cells, may play a crucial role in regulating the inflammatory response. The presence of a strong anti-inflammatory regulatory network, characterized by elevated interleukin-10 (IL-10) and transforming growth factor beta (TGF-β) produced by antigen-presenting cells, and/or regulatory T cells, could help to prevent the cascade of events leading to allergic inflammation. Allergic individuals express lower levels of IL-10; moreover, successful immunotherapy is associated with a sharp increase in IL-10 production by T-cells, and IL-10 increases in atopic children receiving probiotic supplementation (23).

Many chronic disease conditions, such as COPD, heart failure, renal failure, inflammatory bowel disease and rheumatoid arthritis are frequently accompanied by chronic loss of body weight, fat mass, lean tissues such as muscle and/or bone mass, and muscle strength, as well as by anorexia (loss of appetite), general loss in performance status, leading to reduction in daily activities and inherent loss of quality of life. The weight loss is associated with extensive wasting of energy stores of fat, skeletal muscle, and with elevated lipolysis and proteolysis. Although a similar syndrome of weight loss is seen in patients with cancer, especially patients of some tumor types, the underlying mechanism is distinct: cancer cachexia is thought to be due in part to tumor-produced proteolytic and lypolytic humeral mediators (24, 25), whereas the loss of fat and lean tissues in the aforementioned inflammatory conditions other than cancer is not caused by tumor-derived substances, but apparently associated with the inflammatory process as such. Also in sarcopenia, the loss of muscle mass associated with old age, chronic excessive inflammation is now thought to play a crucial role.

Sickness behavior is defined as a symptom complex that accompanies the response to infection and is characterized by fatigue, anorexia, weight loss, sleep disorders, and loss of interest in usual activities (26). It has been known for a number of years that infusions of cytokines including tumor necrosis factor alpha (TNF-α), IL-12 and interferon gamma (IFN-γ) induce fatigue and sleepiness as a major side effect (27). Studies in experimental animals have shown that peripheral and central injections of lipopolysaccharide (LPS), a cytokine inducer, and recombinant pro-inflammatory cytokines, induce sickness behavior (26). Patients with pathologically increased daytime sleepiness and fatigue had elevated levels of circulating TNF-α (28). Also, TNF-α (29) and IL-1β (28) were implicated in the etiology of chronic fatigue syndrome (CFS). At the molecular level, sickness behavior is thought to be mediated by an inducible brain cytokine compartment that is activated by peripheral cytokines via neural afferent pathways. Centrally produced cytokines act on brain cytokine receptors that are similar to those characterized on peripheral immune and non-immune cells. At present, no effective therapy for sickness behavior and fatigue exists.

Aberrant immune responses also play a role in many other disorders including, amongst others, auto-immune disorders, peri-operative immunosuppression (which is considered as a major problem in cancer because of the increased risk of metastatic spread of cancer cells via the vascular system), in acquired immune deficiency syndrome (AIDS), and other similar conditions. Moreover, anti-inflammatory and immunosuppressive drugs may cause serious side effects such as limited resistance to infections. Such side effects are partly due to the fact that such drugs exploit certain mechanisms in the human body, but are not part and parcel of the evolutionary physiological processes and pathways of synthesis and breakdown in the body.

In many inflammatory disorders (including amongst others COPD, IBD and RA, as discussed above), oxidative stress plays an important role. In healthy humans, reactive oxygen species are constantly generated, but this process is well regulated by scavenging abundant radicals via the antioxidant defense system. However, in patients with active diseases or pro-inflammatory conditions, such as COPD, IBD, RA and others, an increased and unbalanced production of reactive oxygen species occurs. This phenomenon is called oxidative stress. Amongst many different effects, oxidative stress induces lipid peroxidation in biomembranes, leading to increased Ca²⁺-influx, changes in receptors, etc. Furthermore, oxidative stress leads to oxidation of SH-moieties, not only in reduced glutathione (GSH) but also in membrane-bound Ca²⁺-ATPases (which provide an ATP-dependent active pumping system). Upon exposure to oxidative stress, the intracellular concentration of ATP decreases (30). As a result, periods of oxidative stress are often followed by an increase in intracellular Ca²⁺ levels, which can result in cell death. Oxidative stress and inflammation may also occur as a consequence of treatments such as radiotherapy and chemotherapy in cancer patients, and may cause damage to healthy tissues and side effects such as intestinal problems, dry mouth, etc.

New Effects of ATP and Novel Concepts Regarding the Physiological Role of ATP in Immunity and Inflammation

As indicated above, we found in accordance with the present invention that ATP inhibits inflammation by inhibiting the excessive inflammatory response to an external insult such as endotoxin (LPS) or phytohaemagglutinin (PHA). To that end, a model was used (see experimental section) that simulates the in vivo situation, i.e. whole blood ex vivo. In this model, ATP exerts marked anti-inflammatory effects. The results show for the first time that ATP in whole blood (i.e. in a model which comes close to the in vivo situation, in contrast to studies in isolated blood cells or cell lines which are far away from the in vivo situation) inhibits the inflammatory response to a strong inflammatory insult such as LPS and PHA, thus modulating the cytokine production in whole blood. The observed response is highly consistent in different subjects.

In addition, we found in accordance with the present invention that ATP inhibits excessive inflammation by inhibiting the inflammatory response to an external insult such as LPS and PHA even under circumstances of oxidative stress. To that end, in the same model as mentioned above, i.e. whole blood was incubated ex vivo with LPS and PHA in the presence of ATP and hydrogen peroxide (H₂O₂). In this model, despite the presence of H₂O₂, ATP exerts similar anti-inflammatory effects as described above. Details of the experiment will be described in the experimental section below. The results show for the first time that ATP inhibits the inflammatory response to a strong inflammatory insult such as LPS and PHA in the presence of oxidative stress, by modulating the cytokine production in whole blood. As before, the observed response is highly consistent in different subjects.

Furthermore, it was found in accordance with the present invention that the anti-inflammatory effects of adenosine, AMP and ADP are less marked than those of ATP in the same model. It was also found that the effects of ATP can be at least partly mimicked by incubating whole blood ex vivo with P2 receptor agonists, such as 2-MeS-ATP, instead of ATP, in the same model as described above. Results of this experiment show that 2-MeS-ATP mimics the attenuation of inflammatory response to a strong inflammatory insult such as LPS and PHA. Thus, these combined results indicate that the anti-inflammatory effects of ATP are stronger than those of adenosine and, at least in part, independent from the previously described effects of adenosine.

It is further expected in the framework of the present invention that ATP reduces the intestinal permeability induced by NSAIDs in the small intestine of human subjects, as assessed by the lactulose/rhamnose (L/R) intestinal permeability test. Specifically, results show that the urinary lactose/rhamnose excretion ratio after ingestion of ATP and indomethacin is lower than after ingestion of indomethacin alone. The effect of ATP is believed to be stronger than that of adenosine, the main breakdown product of ATP.

It is essential to note a major difference between the long-term favourable effects of ATP according to the present invention and the known immediate bronchoconstrictive effects of inhaled adenosine and ATP mentioned above. Essentially, according to the present invention:
ATP is given as an intravenous infusion, in contrast to inhalation as described in the literature;
ATP is started at a low infusion rate (e.g. 20 mcg/kg.min) which is slowly increased at small steps of e.g. 10 mcg/kg.min, as will be described below, and
The maximally tolerated dose of ATP is determined individually in each subject.

Generally speaking, according to the experience of the inventors, pulmonary side effects of ATP infusion in patients with asthma and/or COPD may often occur at lower infusion rates than in subjects without these diseases, so that the maximally tolerated dose of ATP in patients with asthma and/or COPD may be lower compared to subjects without pulmonary diseases; however, surprisingly, this does not preclude the long-term efficacy of ATP in alleviating in COPD patients, amongst others, pulmonary symptoms such as shortness of breath and dyspnoea, and in improving, amongst others, pulmonary function, daily functioning, etc.

Although the inventors do not wish to be bound to any theory, it is believed based on their experiments that the above effect, as well as other effects described above, is caused to a great extent by specific and concerted stimulation of different P2 purinergic receptors by ATP, possibly in combination with indirect effects through P1 purinergic receptors. In addition, ectoenzymes such as ecto-ATPase may act as signaling molecules which, upon stimulation by ATP, inter alia regulate effector functions of immune cells such as lymphocytes. Mechanisms of ATP-induced favorable effects may inter alia include regulation of membrane pore formation; cyclic AMP- and/or calcium²⁺ mediated pathways; signal transduction through inositol phosphate and related compounds; transcription pathways related to nuclear factor kappa B (NFκB); inhibition of poly(ADP-ribose) polymerase (PARP), mitochondrial pathways; etc. etc.

In conclusion, ATP generally is not simply an anti-inflammatory agent, but rather an essential immuno-modulating agent which plays a central and essential role in controlling the inflammatory response and immune competence within the mammalian body. Thus, in situations where a pro-inflammatory stimulus is needed, for instance in immune-compromised or immunosuppressive states, etc. extracellular ATP plays a central role in evoking inflammatory response. In contrast, in situations of excessive inflammatory response, such as after trauma or surgery, in inflammatory pain conditions, in rheumatoid arthritis, in autoimmune disorders, atopic disease, etc., extracellular ATP helps in dampening or terminating the inflammatory process, amongst others by inducing apoptosis of inflammatory cells such as cytotoxic T-lymphocytes.

When performing these roles, it is concluded that ATP is essentially different from any synthetic compounds in that it is part and parcel of normal physiological processes of synthesis, breakdown, and feedback mechanisms; moreover, synthetic purinergic compounds stimulate only a selection of purinergic receptors, which—in combination with the fact that they are not part and parcel of normal physiological processes of synthesis, breakdown, and feedback mechanisms—explains the vast spectrum of side effects of these compounds, including immunosuppression, excessive inflammation, etc. ATP is superior to such synthetic purinergic compounds in that, upon administration, it neither causes immunosuppression, nor excessive inflammation, nor other unwanted or prolonged side effects. The effects of ATP within the framework of the present invention are also superior to those of adenosine, since adenosine merely stimulates P1 purinergic receptors.

It is also expected that ATP is involved in the regulation of the aberrant immune response in atopic and auto-immune diseases, possibly through the newly discovered regulatory T cells.

A practical application of these findings which form part of the present invention is the use of, for example, varying ATP infusion rates, at different duration, frequency, dosage, route of administration, etc. in order to achieve differential effects on different immune-related effects. For instance, it has been found that the optimal dosage of ATP for the treatment of fatigue is different from the optimal dose for increasing muscle mass.

New Uses of ATP

The findings indicate that, in addition to the previously described anabolic properties of ATP, ATP is potentially useful as an immuno-modulating, partly anti-inflammatory, and tissue-protecting drug under varying conditions of oxidative stress and inflammation, as well as under varying conditions and disorders related to immuno-incompetence and immunosuppression.

In preventing and treating certain clinical conditions and diseases, ATP can be inter alia used in accordance with the present invention in the following conditions:
Intestinal inflammation and/or intestinal damage and similar conditions, including amongst other things the inflammation and/or damage induced by NSAIDs or other insults or substances (e.g. alcohol, exercise, smoking, etc.) in healthy and diseased subjects, diarrhea, obstipation, irritable bowel syndrome and different forms of inflammatory bowel disease.
Rheumatoid arthritis and similar conditions (as outlined above).
Chronic Obstructive Pulmonary Disease and similar conditions (as outlined above). We describe herein long-term favourable effects of low-dose ATP infusion in contrast to the state of the art which only relates to immediate bronchoconstrictive effects induced by inhalation of adenosine or ATP.
In patients with cancer, treatment with ATP in combination with (i.e. before, during or after) radiotherapy, chemotherapy or surgery, will reduce the inflammation caused by these treatments in healthy tissues, leading to, amongst other effects, a reduction in short and long term physical side effects such as dry/sore mouth, obstipation, and fatigue; an increased appetite; an improved nutritional status, and prolonged survival.
In combination with anti-cancer treatment such as radiotherapy and/or chemotherapy, treatment with ATP will lead to improved tumour control (tumour response/time to progression) and prolonged survival;
Also, in accordance with the present invention, ATP can be usefully applied in the prevention and treatment of conditions related to the neurological and mental state and functioning, such as: to prevent and treat different types of fatigue, including burn out; to improve sleep quality and prevent or treat sleeping difficulties; to enhance concentration or resolve problems of concentration; to prevent and treat dementia, depression, and/or anxiety; to prevent and treat other mood-related conditions such as inter alia worrying, despair, irritability, tension, stress; disorders related to balance such as dizziness; fibromyalgia; sore muscles; anergy; decreased sexual interest, or similar conditions; sickness behaviour; conditions related to temperature regulation such as shivering; etc.
Furthermore, ATP can be usefully applied in the prevention and treatment of atopic diseases and allergies, such as atopic dermatitis, rhinitis, vernal conjunctivitis and/or asthma;
ATP can be further usefully applied in other diseases and conditions with an elevated or aberrant inflammatory response, for example, peri-operative inflammatory response, trauma, endotoxaemia in healthy and diseased subjects, systemic inflammatory distress syndrome (SIRS), acute and chronic cardiovascular diseases, atherosclerosis, heart failure, syndrome X, endocrine pancreatic disorders, obesity, anorexia, wasting conditions such as cachexia and sarcopenia with loss of lean body mass, muscle mass, muscle strength and/or fat mass, osteoporosis, fibromyalgia, infectious diseases, and inflammatory pain syndromes, auto-immune disorders, skin disorders, peri-operative immunosuppression, AIDS, and other similar conditions, and in the treatment of unwanted: side effects of anti-inflammatory and immunosuppressive drugs, for instance the immuno-incompetence or limited resistance to infections as a consequence of administration of these drugs.

Based on our findings further research programs relating to the use of ATP for the prophylaxis and treatment of certain diseases and disorders have been developed by the present inventors which are now in progress. We summarize below some beneficial effects of ATP which have been found or are expected in treating certain conditions in accordance with the present invention (based on results obtained so far and/or knowledge gained by the inventors from earlier results and observations).

Intestinal inflammatory conditions (including inflammation induced by insults or substances such as NSAIDs, alcohol, exercise and smoking):

Reduced intestinal permeability;

Down-regulation of local pro-inflammatory mediators including cytokines (e.g. TNF-α), positive acute phase proteins and transcription factors;

Up-regulation of local anti-inflammatory mediators including cytokines (e.g. IL-10) and negative acute phase proteins;

Reduction of complaints related to intestinal motility such as, for instance, diarrhea, obstipation, and irritable bowel syndrome.

Inflammatory Bowel Disease:

Reduced intestinal permeability.

Down-regulation of local and systemic pro-inflammatory mediators including cytokines (e.g. TNF-α), positive acute phase proteins and transcription factors;

Up-regulation of local and systemic anti-inflammatory mediators including cytokines (e.g. IL-10) and negative acute phase proteins;

Reduction of erythrocyte sedimentation rate;

Reduction of disease activity and disease progression;

Improvement of disease symptoms such as diarrhea/constipation, and/or pain,

Reduced loss of bone mineral mass;

Inhibition of loss in body weight, fat mass, muscle mass and bone mineral mass;

Increased appetite;

Amelioration of fatigue;

Increased activity level;

Improvement of daily functioning;

Improvement of quality of life.

Rheumatoid Arthritis:

Down-regulation of pro-inflammatory mediators including cytokines (e.g. TNF-α), positive acute phase proteins and transcription factors;

Up-regulation of anti-inflammatory mediators including cytokines (e.g. IL-10) and negative acute phase proteins;

Reduction of erythrocyte sedimentation rate;

Reduction of disease activity and disease progression;

Reduction of joint swelling;

Reduction of pain;

Reduction of cartilage collagen damage and bone destruction;

Inhibition of loss in body weight, fat mass and muscle mass;

Increased appetite;

Amelioration of fatigue;

Increased activity level;

Improvement of daily functioning;

Improvement of quality of life.

Chronic Obstructive Pulmonary Disease:

Improved lung function, e.g. forced expiratory volume in 1 s (FEV1);

Amelioration of dyspnoea and shortness of breath;

Increase in physical activity and physical working capacity;

Amelioration of disease symptoms;

Inhibition of loss of weight, lean body mass, muscle mass and fat mass;

Improved muscle strength and muscle function including, amongst others, muscle aerobic metabolism, muscle energy status, and muscle cell differentiation;

Down-regulation of local and systemic pro-inflammatory mediators including cytokines (e.g. TNF-α), positive acute phase proteins and transcription factors;

Up-regulation of local and systemic anti-inflammatory mediators including cytokines (e.g. IL-10) and negative acute phase proteins;

Amelioration of fatigue;

Increased appetite;

Increased activity level;

Improvement of daily functioning;

Improvement of quality of life.

Patients Suffering from Cancer:

ATP in combination with (i.e. before, during or after) radiotherapy, chemotherapy or surgery treatment, will attenuate the inflammation in healthy tissues, leading to, inter alia, a reduction in short term and prolonged physical side effects related to epithelial and other damage, such as dry/sore mouth, swallowing complaints, intestinal complaints, diarrhea, obstipation, or long term intestinal damage from these treatments; a reduction in fatigue, increased appetite, an improvement in nutritional status, muscle mass and muscle strength, and prolonged survival in cancer patients.

Improved tumor control (tumor response/time to progression), in particular in combination with anti-cancer drugs (radiotherapy and/or chemotherapy), again contributing to prolonged survival in cancer patients.

Atopic Diseases:

Prevention and/or alleviation of allergic symptoms such as wheezing, coughing, rhinitis and asthmatic symptoms;

Down-regulation of local and systemic pro-inflammatory mediators including cytokines (e.g. IL-12) and immunological markers associated with atopic disposition or phenotype (e.g. IgE);

Up-regulation of local and systemic anti-inflammatory mediators including cytokines;

Regulation of the immune response by skewing the immune balance from an aberrant and/or excessive Th-2 response towards a Th-1 response.

Conditions such as Fatigue, Fibromyalgia, Burn-Out and Depression:

Amelioration of disease symptoms;

Amelioration of fatigue;

Increase in physical activity and physical working capacity;

Relief of depression;

Relief of anxiety;

Relief of tension;

Improved sleep quality;

Improved learning capacity, concentration and memory storage;

Amelioration of quality of life;

Down-regulation of local and systemic pro-inflammatory mediators including cytokines (e.g. TNF-α);

Up-regulation of local and systemic anti-inflammatory mediators including cytokines.

Furthermore, in preventing and treating immunosuppressive disorders such as low resistance to infections, or immuno-incompetence due to the treatment with anti-inflammatory or immunosuppressive drugs, the immunoregulatory effects of ATP may help to prevent or reduce the severity of unwanted side effects. In immune deficiency diseases, ATP may aid in increasing immune competence. In preventing and treating auto-immune disorders, ATP may help in restoring deregulated immune responses, inter alia by downregulating the inflammatory response and by skewing the immune balance from an aberrant and/or excessive Th-1 response towards a Th-2 response.

In general, it is expected that these effects of ATP will not only aid in the primary, secondary and tertiary prevention and treatment of diseases and disorders, thus reducing the burden and suffering of patients, but also contribute to lowering health care costs and increasing work participation in some of the aforementioned chronic inflammatory diseases and conditions as well as other immunological disorders, burn out syndrome, etc.

Preparation and Administration of ATP and Compositions Comprising ATP

When applying ATP in accordance with the present invention in mammals, preferably human beings, the medicine is usually and conveniently in the form of a pharmaceutical or nutritional composition, preferably a pharmaceutical composition for oral or parenteral administration. The pharmaceutical composition for parenteral administration is preferably adapted for continuous infusion of ATP, more preferably in an amount up to 150 μg/kg.min for regular administration, the composition further comprising a pharmaceutically acceptable carrier. The amount of ATP in nutritional compositions (or food supplements) is preferably subdivided in dosages of up to 25 g/day for regular administration.

Pharmaceutical and nutritional compositions comprising ATP can be prepared by any convenient manner which is known to a person skilled in the art. In one preferred embodiment of the invention, a pharmaceutical composition is formulated as the disodium salt of ATP (ATP-Na₂). In another preferred embodiment, a pharmaceutical composition is formulated as a lyophilized preparation of ATP-Na₂.

We have now developed and tested ways and methods to safely administer ATP by intravenous infusion in the setting of private homes, i.e. without direct medical supervision, by a trained nurse. Within the framework of the present invention a training program for nurses is provided to safely prepare and administer ATP solutions by intravenous infusion. In a preferred embodiment of the invention, after one ATP infusion course which is preferably administered under medical supervision, subsequent ATP infusions can be given without medical supervision e.g. in the home setting by a trained nurse. The said training program for nurses has been developed to safely prepare and administer ATP solutions by intravenous infusion. This program has been tested in home care organizations in four different regions within the European Union, demonstrating that this is not dependent on the region or country provided trained nurses supported by a hospital, nursing home, home care organizations or any comparable professional health care organization exists.

In one preferred aspect of the invention, ATP is administered in combination with phosphate in either inorganic, organic or any other form during the same period of time, in subsequent order, or alternating. In particular, Rapaport (9,10) has described that adenosine administered in combination with phosphate inhibited host weight loss of tumor-bearing animals to a similar extent as ATP, whereas adenosine without phosphate was ineffective. Based on this prior art, we expect that administration of nucleosides such as adenosine in combination with inorganic phosphate will also be similarly effective as ATP.

Freeze-drying can be performed in any conventional way which is known to a person skilled in the art. In a preferred embodiment of the invention, freeze-drying is performed in a KLEE freeze dryer essentially according to the following procedure:
1. Sterilized standard freeze-drying stoppers are pre-treated for 24 hours at 110° C. to remove moisture;
2. Solutions of mannitol in the range of 0.01% to about 25%, preferably 1.5 to 6%) or HES in the range of 0.01% to about 25%, preferably 1.5 to 3%, are prepared with distilled water (other filler(s) known in the art can be used alternatively);
3. ATP is added to these solutions (preferably about 1 g/10 ml);
4. Sterilized 3 ml freeze-drying vials are filled with 0.50 ml of a solution containing ATP, using a calibrated Gilson pipet;
5. Vials are stoppered with standard rubber stoppers;
6. Vials are stored at ambient temperature for up to 1 hour;
7. Vials are placed on shelves of the freeze-dryer which are precooled to −38° C.;
8. Freezing of the solutions is performed for 45 min on the precooled shelves;
9. The freeze-drying cycle is then started;
10. After lowering the chamber pressure in the freeze-dryer to 8×10⁻² mbar, the temperature is kept at −18° C. during primary drying phase;
11. During the secondary drying phase, the process is controlled using pressure raise testing.

In contrast to crystalline ATP and ATP in solution, the lyophilized ATP preparation is stable at room temperature for at least 1 to 3 years. It can be easily dissolved in saline and thus the infusion solution can be prepared freshly by a trained nurse. In this way, it is logistically feasible and safe to administer ATP in the setting of a private home, nursing home, etc. by a trained nurse, without need for medical intervention.

In another preferred embodiment of the invention, ATP is administered as a series of about 1 to 20 intravenous infusions at intervals of about 1 to 4 weeks.

In order to determine the tolerance for ATP as well as the maximally tolerated dose of ATP, the first ATP infusion is preferably administered under medical supervision, usually in an in- or outpatient setting. Subsequent infusions can either be started at the hospital day care centre, at private homes, nursing homes, etc. according to a standardized protocol. Our experience shows, for the first time, that it is feasible and safe to administer subsequent ATP infusions in the home setting. In a total of over 60 home infusions in cancer patients, no serious side effects grade 3-4 on the WHO Common Toxicity Criteria scale occurred. No hospital admissions were necessary.

The preparation may be given as an intravenous infusion of 5-150 mcg of ATP etc. per kg body weight per minute, at a frequency varying from continuous infusion to low frequency (e.g. once per year). A suitable infusion time and frequency is, for example, 8-12 hours or 24-30 hours of ATP infusion once per week or once per 2-8 weeks. Another suitable frequency is, for example, 1 minute to 4 hours every day for a certain period, with or without days of interrupting the infusions. Instead of intravenous infusion, other routes of administration may be preferred: intraperitoneal, subcutaneous, oral, topical, nasal, sublingual, etc.

In a further preferred embodiment of the invention, intravenous infusion of ATP is initiated at an infusion rate ranging from about 5 to about 40 μg/kg.min, preferably of about 20 μg/kg.min which is subsequently increased by steps ranging from about 5 to about 20 μg/kg.min, preferably of about 10 μg/kg.min every 5-30 min., preferably about 10 min. If side effects appear, the infusion rate is reduced in steps preferably of about 10 μg/kg.min every 5-30 min (preferably about 10 min) to the dose where side effects have fully disappeared. This dose is the maximally tolerated dose, which essentially has to be determined individually in each subject.

According to the present invention, the frequency, duration and rate of ATP infusion may be varied in order to achieve desired specific effects. For instance, in one preferred aspect of the invention, when aiming at increasing muscle strength, a dosage of about 75 μg/kg.min may be applied, whereas a dosage between about 40 to 60 μg/kg.min may be given when aiming at ameliorating shortness of breath, constipation, fatigue or quality of life. Variations in dosage and/or concentration of ATP, further ingredients of the composition, frequency, etc. depend on several individual factors of the individual to be administered, such as age, sex, condition of the individual, and are usually determined on an individual basis by a physician or other skilled person. The ATP solution may contain ATP in the form of one or more salts, e.g. mono- or di-Na-ATP, Mg-ATP or the combination of ATP etc. with MgCl₂, preferably in conjunction with a pharmaceutically acceptable carrier or vehicle and/or other ingredients which are known in the art.

In accordance with the present invention ATP and/or derivatives can be applied in parenteral and enteral nutrition, alone or in combination with specific compounds comprising those mentioned within this application. The preparation of such compositions is well known to people skilled in the art and can be optimized in a routine way without exerting inventive skill and without undue experimentation. The dosage and frequency of administration depends inter alia on well-known factors, such as the weight of the individual to be administered, age, sex, condition, etc., and will usually be determined by a physician or other person skilled in the art.

Other substances may be given simultaneously in the same pharmaceutical or nutritional preparation which comprises ATP. Another possibility is that various treatment schedules are developed in which administration of ATP and other components may be given during the same period of time, in subsequent order, or alternating, etc. Such other compounds include, for example, phosphate in either inorganic, organic or any other form; n-3 fatty acids such as eicosapentaenoic acid (EPA), docosahexaenoic acid (DHA) and/or alpha-linolenic acid, preferably administered as triacylglycerol, but also as free fatty acids or esters, for example ethyl esters, if desired; creatine; one or more amino acids, such as: cyst(e)ine, preferably as N-acetyl cysteine (NAC), but also other cyst(e)ine derivatives; arginine; glutamine; glutamate; and/or other amino acids; carbohydrates, such as ribose and others; antioxidant vitamins such as vitamin C, vitamin E and others; other antioxidants such as carotenoids, flavonoids, isoflavonoids, phyto-estrogens, and others; minerals and trace elements such as selenium, calcium, magnesium, and others; nutrients, non-nutrients, pharmacological compounds; and the like.

The ATP-containing pharmaceutical compositions which are useful for the purpose of the present invention may additionally comprise one or more substances selected from the group of stimulants, hormones, analogues of such hormones, phyto-hormones, analogues of such phyto-hormones, or other pharmacological compounds of choice, which are all within the realm of a person skilled in the art based on his knowledge, experience and/or experimenting without inventive effort.

Experimental Section

To demonstrate the marked anti-inflammatory effects of ATP a model was used that simulates the in vivo situation, i.e. whole blood ex vivo.

Experiment 1

Methods

For the first experiment, purified phyto-haemagglutinin HA16 (PHA) and E. Coli 0.26: B6 lipopolysaccharide (LPS) were from Murex, Dartford, UK and Sigma Chemical Co, St. Louis, USA, respectively. Human TNF-α (7300 pg/ml) was obtained from CLB/Sanquin, The Netherlands. RPMI 1640 medium containing L-glutamine was obtained from Gibco, UK. Adenosine-5′-triphosphate disodium salt (ATP) was purchased from Calbiochem, USA.

Blood was collected from 8 healthy subjects in heparin containing vacutainer tubes (Vacutainer, Becton-Dickinson, 170 I.U). Pilot experiments showed that storage time (1-4 h) and temperature (4 and 20° C., respectively) had no effect on LPS/PHA-stimulated TNF-α and the IL-10 release from whole blood. Whole blood was aliquoted into 24-well sterile plates and diluted 1:4 with RPMI 1640 (supplemented with L-glutamine). To induce cytokine production, PHA and bacterial LPS were added at 1 μg/ml and 10 μg/ml final concentration respectively. After adding the concentrated solutions of ATP and the stimulants the plates were incubated in 5% CO₂ at 37° C. for 24 h. After the incubation cell-free supernatant fluids were collected by centrifugation (6000 rpm, 10 min at 4° C.) and stored at −20° C. until tested for presence of cytokines.

All incubations were performed in duplicate. ATP was dissolved in RPMI 1640 culture medium, at a final concentration of 1-300 μM, and blood pre-incubated with ATP at 5% CO2 at 37° C. for 30 min before stimulation with LPS en PHA.

All cytokines were quantified by means of PeliKine Compact human ELISA kits (CLB/Sanquin, The Netherlands), based on appropriate and validated sets of monoclonal antibodies. Assays were performed as follows. Monoclonal antibodies specific for each component were pre-coated overnight at room temperature into 96-well polystyrene microtiter plates. Standards and samples were given into the wells and then incubated for 1 h at room temperature. The antibody on the microtiter plate then captured the cytokine present in a measured volume of sample or standard, and non-bound material was removed by washing. Subsequently, a biotinylated second monoclonal antibody for each of the components was added and incubated for 1 h at room temperature. Following washing to remove unbound antibody-enzyme reagents, horseradish peroxidase (HRP) conjugated streptavidin, which binds onto the biotinylated side of the cytokine complex, was added to the wells and incubated for 30 min at room temperature. After removal of non-bound HRP conjugate by washing, the substrate solution was added to the wells and incubated for 30 min at room temperature. Color development was stopped by addition of sulfuric acid and the intensity of the color was measured by a microtiter plate reader (absorbance at 450 nm). The absorbance was transformed to cytokine concentrations (ng/L) using the standard curve. The sensitivity for TNF-α, IL-10 and IL-6 was respectively 4-6 ng/L, 3-5 ng/L and 0.5-1 ng/L.

Statistical significance of change in cytokine release by different ATP concentrations relative to no ATP as a reference was determined using Student's paired t test. P-values <0.05 were considered statistically significant.

Results

Addition of LPS/PHA in the absence of ATP caused production of high measurable quantities of TNF-α, IL-6 and IL-10 in all blood samples. Blood cytokine concentrations were low in control (i.e. not stimulated) samples and increased significantly under LPS+PHA stimulation.

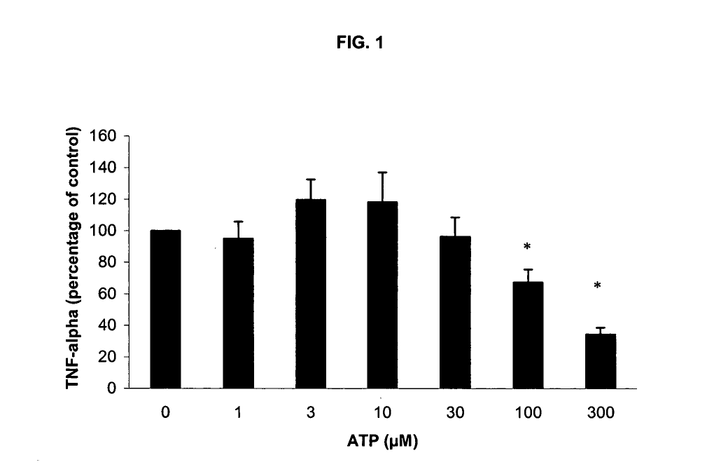

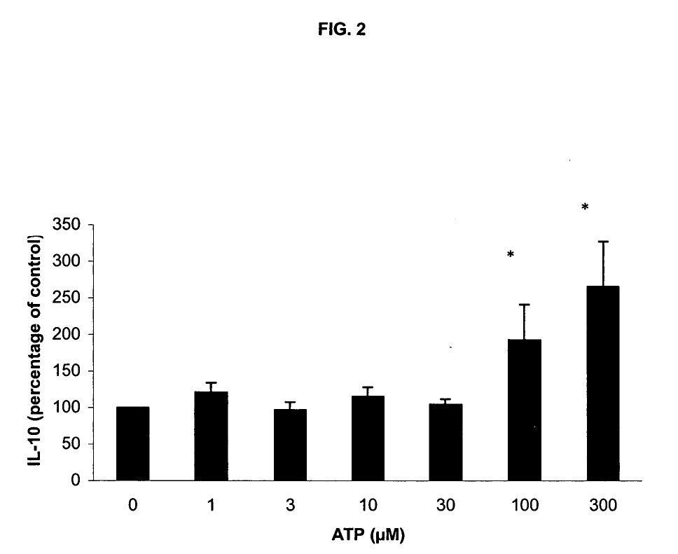

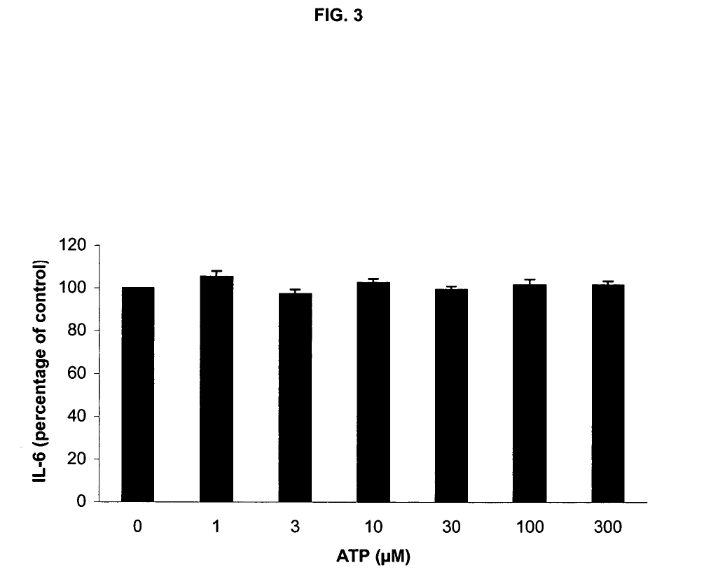

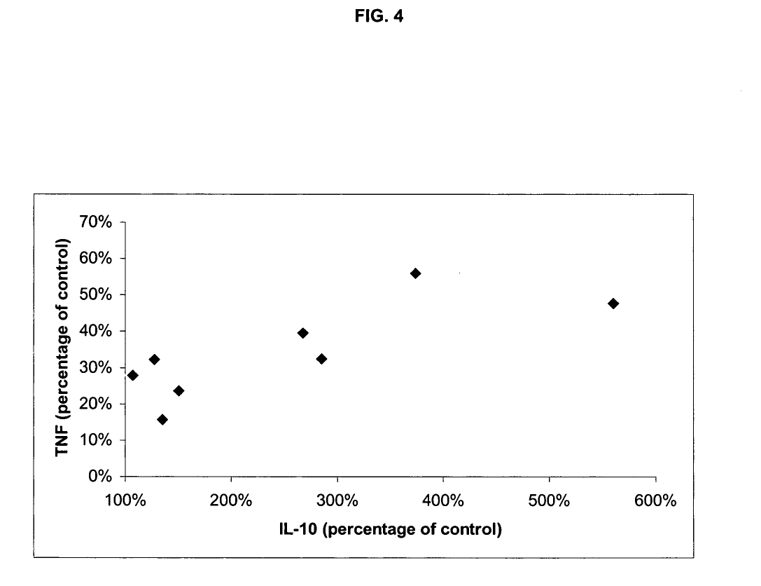

First, we observed that when blood pre-incubated with ATP at 5% CO₂ at 37° C. for 30 min before stimulation with LPS en PHA, a dose-dependent inhibition of the release of the pro-inflammatory cytokine TNF-α in LPS-PHA stimulated whole blood at 100 and 300 μM ATP was observed (FIG. 1). At 300 μM ATP, a 65% inhibition of the TNF-α production was found in stimulated whole blood. We then examined whether ATP could increase the production of the anti-inflammatory cytokine IL-10. As shown in FIG. 2, ATP increased the release of IL-10 in LPS/PHA stimulated whole blood at 100 and 300 μM ATP; at 300 μM of ATP, we found a 62% stimulation of the IL-10 production. Finally, we tested the effect of ATP on the production of IL-6; as shown in FIG. 3, ATP failed to significantly alter the production of this cytokine. There was a significant relationship between the inhibitive effect of ATP on TNF-α release and the stimulative effect of ATP on IL-10 release in LPS-PHA stimulated whole blood (FIG. 4).

Experiment 2

Methods

Whole blood of 8 healthy subjects was collected as described for Experiment 1, pretreated with 1 or 10 mM H₂O₂ and ATP at concentrations of 1-300 μM for 30 minutes, and then incubated as in experiment 1 with LPS/PHA for 24 hours.

Results

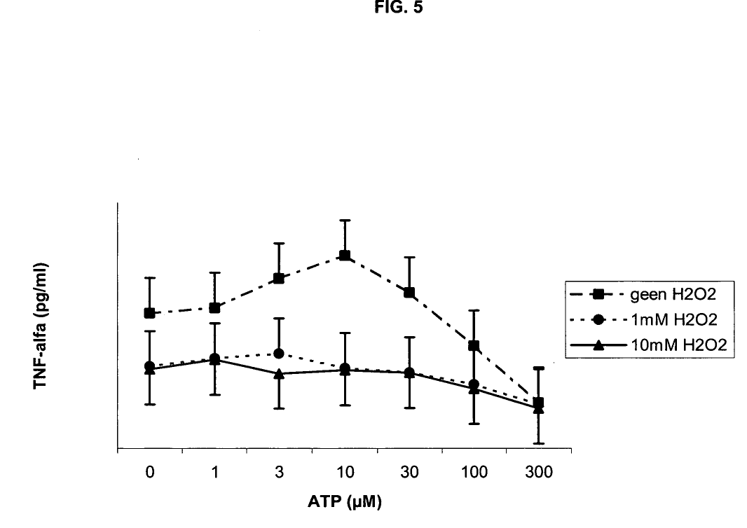

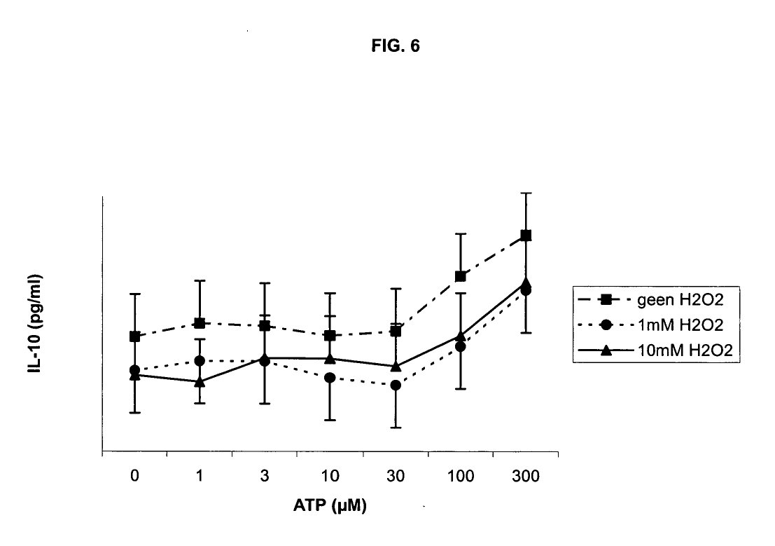

Incubation with LPS/PHA under these conditions without ATP induced a strong release of TNF-α, IL-6 and IL-10. As in Experiment 1, addition of ATP induced a dose-dependent reduction in TNF-α production at 100 and 300 μM ATP (FIG. 5). Also, a significant, dose-dependent rise in IL-10 release was observed (FIG. 5). Again, no effect on IL-6 release was detected (FIG. 6).

Experiment 3

Methods

Blood is collected as described for Experiment 1. Whole blood is then aliquoted into 24-well sterile plates and diluted 1:4 with RPMI 1640 (supplemented with L-glutamine). To induce cytokine production, PHA and bacterial LPS are added to whole blood at 1 μg/ml and 10 μg/ml respectively. After addition of ATP and stimulants, the plates are incubated in 5% CO₂ at 37° C. for 24 h. Cell-free supernatant fluids are then collected by centrifugation (6000 rpm, 10 min at 4° C.) and stored at −20° C. until tested for presence of cytokines. All incubations are performed in duplicate. ATP, dissolved in RPMI 1640 culture medium, is added to the blood at a final concentration of 1-1000 μM. Blood is pre-incubated with ATP at 5% CO₂ at 37° C. for 30 min before stimulation with LPS+PHA. The agonists are added in the same way as ATP, however their stock solutions are prepared in PBS and further diluted in medium.

Results

Our initial findings indicate that pretreatment of whole blood with ATP is more effective in inhibiting TNFα and stimulating IL-10 production in LPS-PHA stimulated whole blood, than ADP, AMP or adenosine. Furthermore, our initial findings indicate that the inhibition of TNFα release and the stimulation of IL-10 release in whole blood can be at least partly mimicked by pretreating blood with P2 receptor agonists, such as 2-MeS-ATP, instead of ATP. Results of this experiment show that 2-MeS-ATP mimics the attenuation of inflammatory response to a strong inflammatory insult such as LPS and PHA.

The combined results of this experiment indicate that the anti-inflammatory effects of ATP are stronger than those of adenosine and, at least in part, independent from the previously described effects of adenosine

Experiment 4

Methods Intestinal permeability is tested in healthy non-smoking human subjects using the lactulose/rhamnose (L/R) intestinal permeability test. This barrier function test is based on the comparison of intestinal permeation of molecules of different sizes by measuring the ratio of urinary excretion of the disaccharide lactulose and the monosaccharide rhamnose. These two sugars follow different routes of intestinal permeation, i.e., lactulose permeates through the paracellular pathway, whereas rhamnose permeates transcellulary. The experiments are performed as follows: at t=−14 hrs, a Bengmark-type naso-intestinal tube (Flocare, Zoetermeer, The Netherlands) is installed into the stomach. Next, at t=−10 hrs, subjects ingest a capsule of indomethacin (75 mg) immediately followed by administration of either ATP or placebo directly into the subject's duodenum through the inserted tube. At t=−1 hr, after an overnight fast, subjects receive a second dose of indomethacin (50 mg) followed by either ATP or placebo. Then, at t=0, the permeability test is performed as follows: subjects ingest a hyperosmolar drink containing 5 g of lactulose and 0.5 g of L-rhamnose dissolve d in 00 ml water. After ingestion of the hyperosmolar test drink, total urine produced over 5 hours is collected.

Results

It is expected that the urinary concentration ratio of lactulose relative to rhamnose in subjects treated with indomethacin and ATP is lower than is the same ratio in subjects treated with indomethacin only.

### REFERENCES

1. Agteresch H J, Dagnelie P C, van den Berg J W, Wilson J H. Adenosine triphosphate: established and potential clinical applications. Drugs 1999; 58(2):211-32.
2. Agteresch H J, Dagnelie P C, Rietveld T, van den Berg J W, Danser A H, Wilson J H. Pharmacokinetics of intravenous ATP in cancer patients. Eur J Clin Pharmacol 2000; 56(1):49-55.
3. Leij-Halfwerk S, Agteresch H J, Sijens P E, Dagnelie P C. Adenosine triphosphate infusion increases liver energy status in advanced lung cancer patients: an in vivo 31P magnetic resonance spectroscopy study. Hepatology 2002; 35(2):421-4.
4. Rapaport E. Treatment of human tumor cells with ADP or ATP yields arrest of growth in the S phase of the cell cycle. J Cell Physiol 1983; 114(3):279-83.
5. Spungin B, Friedberg I. Growth inhibition of breast cancer cells induced by exogenous ATP. J Cell Physiol 1993; 157(3):502-8.
6. Weisman G A, Lustig K D, Lane E, Huang N N, Belzer I, Friedberg I. Growth inhibition of transformed mouse fibroblasts by adenine nucleotides occurs via generation of extracellular adenosine. J Biol Chem 1988; 263(25):12367-72.
7. Lasso de la Vega M C, Terradez P, Obrador E, Navarro J, Pellicer J A, Estrela J M. Inhibition of cancer growth and selective glutathione depletion in Ehrlich tumour cells in vivo by extracellular ATP. Biochem J 1994; 298(Pt 1):99-105.
8. Obrador E, Navarro J, Mompo J, Asensi M, Pellicer J A, Estrela J M. Glutathione and the rate of cellular proliferation determine tumour cell sensitivity to tumour necrosis factor in vivo. Biochem J 1997; 325(Pt 1):183-9.
9. Rapaport E, Fontaine J. Generation of extracellular ATP in blood and its mediated inhibition of host weight loss in tumor-bearing mice. Biochem Pharmacol 1989; 38(23):4261-6.
10. Rapaport E, Fontaine J. Anticancer activities of adenine nucleotides in mice are mediated through expansion of erythrocyte ATP pools. Proc Natl Acad Sci USA 1989; 86(5):1662-6.
11. Haskell C M, Wong M, Williams A, Lee L Y. Phase I trial of extracellular adenosine 5′-triphosphate in patients with advanced cancer. Med Pediatr Oncol 1996; 27(3):165-73.
12. Haskell C M, Mendoza E, Pisters K M, Fossella F V, Figlin R A. Phase II study of intravenous adenosine 5′-triphosphate in patients with previously untreated stage IIIB and stage IV non-small cell lung cancer. Invest New Drugs 1998; 16(1):81-5.
13. Agteresch H J, Dagnelie P C, van Der Gaast A, Stijnen T, Wilson J H. Randomized clinical trial of adenosine 5′-triphosphate in patients with advanced non-small-cell lung cancer. J Natl Cancer Inst 2000; 92(4):321-328.
14. Di Virgilio F, Chiozzi P, Ferrari D, Falzoni S, Sanz J M, Morelli A, et al. Nucleotide receptors: an emerging family of regulatory molecules in blood cells. Blood 2001; 97(3):587-600.
15. El Moatassim C, Dornand J, Mani J C. Extracellular ATP and cell signalling. Biochim Biophys Acta 1992; 1134(1):31-45.
16. Fredholm B B. Purines and neutrophil leukocytes. Gen Pharmacol 1997;28(3):345-50.
17. Carty E, Rampton D S. Evaluation of new therapies for inflammatory bowel disease. Br J Clin Pharmacol 2003; 56(4):351-61.
18. Kruidenier L, Kuiper I, Lamers C B, Verspaget H W. Intestinal oxidative damage in inflammatory bowel disease: semi-quantification, localization, and association with mucosal antioxidants. J Pathol 2003; 201(1):28-36.
19. Karatas F, Ozates I, Canatan H, Halifeoglu I, Karatepe M, Colakt R. Antioxidant status & lipid peroxidation in patients with rheumatoid arthritis. Indian J Med Res 2003; 118:178-81.
20. Bjarnason I, Hayllar J, MacPherson A J, Russell A S. Side effects of nonsteroidal anti-inflammatory drugs on the small and large intestine in humans. Gastroenterology 1993; 104(6): 1832-47.
21. Pelleg A, Schulman E S. Adenosine 5′-triphosphate axis in obstructive airway diseases. Am J Ther 2002; 9(5):454-64.
22. Adriaensen D, Timmermans J P. Purinergic signalling in the lung: important in asthma and COPD? Curr Opin Pharmacol 2004; 4(3):207-14.
23. Yazdanbakhsh M, Kremsner P G, van Ree R. Allergy, parasites, and the hygiene hypothesis. Science 2002; 296(5567):490-4.
24. Tisdale M J, Russell S T. The ‘cancer cachectic factor’. Support Care Cancer 2003; 11(2):73-8.
25. Russell S T, Tisdale M J. Effect of a tumour-derived lipid-mobilising factor on glucose and lipid metabolism in vivo. Br J Cancer 2002; 87(5):580-4.
26. Maier S F, Watkins L R. Intracerebroventricular interleukin-1 receptor antagonist blocks the enhancement of fear conditioning and interference with escape produced by inescapable shock. Brain Res 1995; 695(2):279-82.
27. Krueger J M, Obal F J, Fang J, Kubota T, Taishi P. The role of cytokines in physiological sleep regulation. Ann N Y Acad Sci 2001; 933:211-21.
28. Cannon J G, Angel J B, Abad L W, Vannier E, Mileno M D, Fagioli L, et al. Interleukin-1 beta, interleukin-1 receptor antagonist, and soluble interleukin-1 receptor type II secretion in chronic fatigue syndrome. J Clin Immunol 1997; 17(3):253-261.
29. Moss R B, Mercandetti A, Vojdani A. TNF-alpha and chronic fatigue syndrome. J Clin Immunol 1999; 19(5):314-316.
30. Bast A. Oxidative stress and calcium homeostasis. In: Halliwell B, Ol A, editors. DNA and free radicals. New York: Ellis Horwood; 1993. p. 95-108.

1. Use of ATP for the manufacture of a medicament for exerting a pharmacological effect when administered to a mammal, selected from the group consisting of:
1°. modulating inflammation by inhibiting the inflammatory response to a strong external insult, such as endotoxin (LPS) and/or phytohaemagglutinin;
2°. exerting said inhibitory effect on inflammatory response to an external stimulus even under conditions of oxidative stress,
3°. exerting a local immuno-modulating and anti-inflammatory effect in the intestine, thus preventing intestinal damage induced by a non-steroid anti-inflammatory drug (NSAIDs),
4°. exerting an immuno-modulating and anti-inflammatory effect in human intestinal cells in vitro,
5°. alleviating pulmonary symptoms, such as shortness of breath and dyspnoea, in patients suffering from an obstructive pulmonary disease, and
6°. exerting a favourable clinical effect with respect to a mental or neurological disorder or aberrant condition.

2. Use of ATP for the manufacture of a medicine having an preventive or curative activity when administered to a mammal, selected from the group consisting of:
1°. tissue-protecting activity which attenuates excessive inflammation under varying conditions of oxidative stress and inflammation;
2°. immune-stimulating activity under varying conditions related to immune-incompetence and immuno-suppression;
3°. immuno-modulating activity normalizing the Th1/Th2 balance in aberrant conditions of aberrant Th2-skewed immune response, such as atopic diseases and asthma, as well as in conditions of aberrant Th1-skewed response, such as auto-immune disorders;
4°. modulating and normalizing aberrant mental and neurological states and diseases.

3. Use of ATP according to claim 1, wherein the medicine is for preventing or treating at least one of intestinal inflammatory condition, intestinal damage, and inflammatory bowel disease.

4. Use of ATP according to claim 1, wherein the medicine is for preventing or treating rheumatoid arthritis.

5. Use of ATP according to claim 1, wherein the medicine is for preventing or treating an atopic disease, including asthma.

6. Use of ATP according to claim 1, wherein the medicine is for preventing or treating a condition selected from the group consisting of fatigue, fibromyalgia, burn-out and depression.

7. Use of ATP according to claim 1, wherein the medicine is for preventing or treating a disease or disorder or condition selected from the group consisting of cancer during and after treatment by at least one of surgery, radiotherapy and chemotherapy, neurological and mental diseases/conditions, and another condition of an elevated or aberrant inflammatory response.

8. A method of preventing or treating an invidual for a disease or disorder or condition selected from the group consisting of intestinal inflammation, intestinal damage, rheumatoid arthritis, COPD, cancer during or after treatment by at least one of surgery, radiotherapy, and chemotherapy, a neurological or mental disorder, an atopic disease including asthma, and another condition of elevated or aberrant inflammatory response, which comprises administering to said individual in need thereof a medicine comprising an effective amount of ATP.

9. Use of ATP according claim 1, wherein the medicine is in the form of a pharmaceutical composition or a nutritional composition.

10. Use of ATP according to claim 9, wherein the medicine is in a lyophilized form.

11. Use of ATP according to claim 2, wherein the medicine is for preventing or treating at least one of intestinal inflammatory condition, intestinal damage, and inflammatory bowel disease.

12. Use of ATP according to claim 2, wherein the medicine is for preventing or treating rheumatoid arthritis.

13. Use of ATP according to claim 2, wherein the medicine is for preventing or treating an atopic disease, including asthma.

14. Use of ATP according to claim 2, wherein the medicine is for preventing or treating a condition selected from the group consisting of fatigue, fibromyalgia, burn-out and depression.

15. Use of ATP according to claim 2, wherein the medicine is for preventing or treating a disease or disorder or condition selected from the group consisting of cancer during and after treatment by at least one of surgery, radiotherapy and chemotherapy, neurological and mental diseases/conditions, and another condition of an elevated or aberrant inflammatory response.

16. Use of ATP according to claim 2, wherein the medicine is in the form of a pharmaceutical composition or a nutritional composition.

17. Use of ATP according to claim 2, wherein the medicine is in the form of a pharmaceutical composition or a nutritional composition.

18. Use of ATP according to claim 8, wherein the medicine is in the form of a pharmaceutical composition or a nutritional composition.
